(19) United States
(12) Patent Application Publication
(10) Pub. No.: US 2014/0094516 A1
(43) Pub. Date: Apr. 3, 2014

(54) METHOD OF UTILIZING AZELAIC ACID ESTERS TO MODULATE COMMUNICATIONS MEDIATED BY BIOLOGICAL MOLECULES

(71) Applicants: Robert T. Streeper, Marion, TX (US); Elzbieta Izbicka, Marion, TX (US)

(72) Inventors: Robert T. Streeper, Marion, TX (US); Elzbieta Izbicka, Marion, TX (US)

(21) Appl. No.: 14/097,854

(22) Filed: Dec. 5, 2013

Related U.S. Application Data
(63) Continuation of application No. 13/449,804, filed on Apr. 18, 2012.
Application No. 13/449,804 is a continuation-in-part of application No. 12/459,338, filed on Jun. 30, 2009, now abandoned.

(51) Int. Cl. A61K 31/23

(52) U.S. Cl. CPC A61K 31/23
USPC 514/547; 435/375

(57) ABSTRACT

The treatment of disease in organisms using Macromolecular interaction modulators and Membrane active immunomodulators, particularly selected azelaic acid esters, individually and in combinations, to modulate communications between biological molecules.

### CROSS-REFERENCE TO RELATED APPLICATION

This application is a continuation of application Ser. No. 13/449,804 which was filed Apr. 18, 2012. application Ser. No. 13/449,804 is a continuation in part of application Ser. No. 12/459,338 which was filed Jun. 30, 2009.

### BACKGROUND OF THE INVENTION

1. Field of the Invention

The present invention relates to the use of various compositions, individually and in combination, for modulating communication between cells that are effected by biological molecules for therapeutic treatment.

2. Description of the Related Art

A Macromolecular interaction modulator (MMIM) is a drug or other molecule that is capable of generally altering the interactions between two or more biological molecules that do not necessarily involve binding of the MMIM to any particular active or allosteric binding site of any of the biological molecules.

A Membrane active immunomodulators (MAIM) is a drug or other molecule that is capable of altering the interactions between two or more biological molecules that constitute some part of the functional apparatus of the immune system that does not necessarily involve binding of the MAIM to any particular active or allosteric binding site of any of the biological molecules.

Azelaic acid is a naturally occurring straight chain, 9 carbon atom saturated dicarboxylic acid obtained by oxidation of oleic acid or by chemical, physical or biological oxidation of free or esterified fatty acids. Azelaic acid is a metabolite of longer chain fatty acids in human bodies. It is found also in small amounts in the urine of normal individuals (Mortensen 1984), and in whole grain cereals and some animal products.

Azelaic acid has been known for many years to possess anti-inflammatory and antimicrobial activity. Azelaic acid inhibits a number of enzymes such as tyrosinase, thioredoxin reductase, and oxidoreductases in the mitochondrial respiratory chain. In addition, azelaic acid is a scavenger of toxic reactive oxygen species and is a potent inhibitor of 5-alpha-reductase.

Azelaic acid has been used clinically for many years in the treatment of acne vulgaris as well as in the treatment of hyperpigmentary skin disorders (Fitton 1991). Azelaic acid has also has recently been studied for the treatment of papulopustular rosacea (Maddin 1999).

While azelaic acid has been used primarily in the treatment of dermatological conditions, because of some of its mechanisms of action, it has further clinical utility in conditions unrelated to the skin. Azelaic acid has been shown to have antiproliferative and cytotoxic action on the following tumor cell lines: human cutaneous malignant melanoma (Zaffaroni et al. 1990), human choroidal melanoma (Breathnach et al. 1989), human squamous cell carcinoma (Paetzold et al. 1989), and various fibroblastic lines (Geier et al. 1986). Azelaic acid may also have utility in the prevention and treatment of skin cancer and solar keratosis. Because of its mechanism of action as a potent inhibitor of 5-alpha-reductase, azelaic acid may be applicable to the treatment and prevention of benign enlargement of the prostate as well as cancer of the breast or prostate and other conditions in which 5-alpha-reductase is implicated in biological process, such as hair loss.

U.S. Pat. Nos. 4,292,326, 4,386,104, and U.S. Pat. No. 4,818,768, (Thornfeldt et al.) describe the uses of azelaic acid as well as other dicarboxylic acids in the treatment of acne and melanocytic hyperpigmentary dermatoses. U.S. Pat. Nos. 4,713,394 and 4,885,282 describe the use of azelaic acid as well as other dicarboxylic acids in the treatment of non-acne inflammatory dermatoses and infectious cutaneous diseases such as rosacea, perioral dermatitis, eczema, seborrheic dermatitis, psoriasis, tinea cruris, flat warts, and alopecia greata. One of Thornfeldt's formulations comprises azelaic acid dispersed in a large proportion of ethanol. Thornfeldt's second formulation comprises a complete dispersion of azelaic acid. U.S. Pat. No. 6,451,773 describes a composition for treating acneiform eruption containing a chitosan having a molecular weight ranging from about 500,000 to about 5,000,000 g/mole and a degree of deacylation greater than 80% and an acid-form active ingredient such as azelaic acid for treating acne. U.S. Pat. No. 6,734,210 discloses the stable salts of azelaic acid with polycations.

Venkateswaran, U.S. Pat. No. 5,549,888, teaches a mixture of active ingredients which includes azelaic acid that is partially solubilized by a glycol and ethanol. Venkateswaran also teaches that the formulation has a pH between 2.5 and 4.0. This low pH is liable to cause skin irritation. Azelaic acid itself causes irritation of the skin due to its acidity.

In the field of pharmacology, and as shown in the prior art (particularly in the citations above), azelaic acid esters (AAEs) have classically been used as, and are considered to be “pro-drugs.” Pro-drugs are an inactive (or significantly less active) form, that are later metabolized in vivo into an active metabolite. In the case of AAEs, the drugs were thought to serve as pro-drugs to the active drug, where the AAE was broken down in the body to release the active drug—azelaic acid. The azelaic acid was considered to be the ultimate agent of activity, not the AAE. Previous uses recognized AAEs as having anti-inflammatory and anti-bacterial properties only, and did not consider or failed to observe that AAEs are capable, without need for the formation of azelaic acid, of modulating the non-covalent intermolecular interactions between biological molecules, or that the use of various AAEs in combination other members of the class can be used to induce a range of biological and medical outcomes.

The present invention represents a new class of pharmaceutical compounds that inhibit intercellular and intracellular molecular communications by a previously unrecognized or unappreciated mechanism of action.

The general modulation of intermolecular interactions by drugs has heretofore not been commonly recognized as important by medical science despite the fact that many drugs exert pharmacological effects in this fashion.

As described herein, AAEs modulate intermolecular interactions of biological molecules as MMIMs or in a narrower sense as MAIMs.

### SUMMARY OF THE INVENTION

In accordance with the present invention, production of compositions and methods for the use of these compositions that involve esters of azelaic acid for the treatment of conditions that have in common the characteristic that part of their etiology or mechanism the operation of intracellular and intercellular signaling mediated by the expression, synthesis, release and recognition of biological molecules that are not beneficial to the overall welfare of the host. The production of certain compositions of matter including esters of azelaic acid that modulate the expression, release, synthesis, recognition and action of biological molecules known to be integral to or involved in signaling through mediators involved in intercellular and intracellular communication processes important in human and other animal diseases and conditions. The application of said AAEs, alone or in various combinations with other pharmacologically active materials that benefit in the amelioration, treatment and cure of a range of diseases mediated by intracellular and intercellular signal transduction molecules.

The present invention discloses new methodologies of utilizing AAEs. These esters of azelaic acid of the present invention have utility in treating or preventing a wide variety of conditions related to the aforementioned mechanisms of action of AAEs.

It is therefore an object of the present invention to utilize the esters pharmacologically as esters and not as pro-drugs that break down to release the acid as the ultimate agent of activity. The esters possess distinct patterns of activity and that while the esters do break down to the acid; the esters themselves are the primary agents of activity.

It is a further object of the present invention to combine various esters together to induce desired biochemical outcomes and, by extension, medical outcomes.

It is still a further object of the present invention to use the esters individually and in combinations to modulate interactions between biological molecules.

Still other objects, features, and advantages of the present invention will become evident to those of ordinary skill in the art in light of the following.

### DETAILED DESCRIPTION OF THE PREFERRED EMBODIMENT

The present invention provides a new method of using esters of azelaic acid to modulate communication between biological molecules for therapeutic treatment.

The data on esters of azelaic acid, and the data on other drugs present in the scientific literature, demonstrate that many molecules act at least in part as MMIMs and/or MAIMs. Some common examples include aspirin which, even though it is a demonstrated inhibitor of cyclooxygenase (COX-1 and COX-2) enzymes, also acts as a MAIM as both published sources and our data clearly demonstrate.

The MAIM activity of aspirin is for instance observed in its ability to control inflammation by mechanisms that do not involve inhibition of COX enzymes. Another commonly used drug that acts in part as a MAIM is paracetamol or acetaminophen. Acetaminophen operates as a COX-2 inhibitor but it has a number of unexplained activities that support its classification as a MAIM.

Another example is cholesterol. Cholesterol is an essential component of all tissues of the body, in particular the brain. Numerous studies however have shown that excessive cholesterol has deleterious health effects. Increasing cholesterol in the plasma membrane of cells has been shown to potentiate inflammatory responses, and depleting cholesterol from the plasma membrane has been shown to damp inflammatory responses. Thus cholesterol acts as a MAIM by supporting proper immune function but in excess it is dangerous.

MMIMs/MAIMs can be considered to fall into the several categories:

Primary MAIMs are those compounds that directly interact with the biological molecules whose activity the MAIMs modulates.

Secondary MAIMs act to alter the composition of the lipid membranes in such a way that the biological molecules associated with it change in activity. Secondary MAIMs may for instance act by sequestration membrane components. One such secondary MAIM is hydroxypropyl-beta-cyclodextrin which sequesters cholesterol from cell membranes with resultant alteration in plasma membrane protein functions.

Tertiary MAIMs are molecules that alter the physiological production of membrane components in such a way as to cause alteration in the activities of membrane associated biological molecules.

Examples of Primary MAIMs include:

Dimethylfumarate/monomethylfumarate and salts;

Epigallocatechingallate;

Monolaurylglycerol;

Docosahexaenoic acid;

Eicosapentaenoic acid;

Omega 3 dietary lipids;

Omega 6 dietary lipids;

Miltefosine,

Edelfosine;

Perifosine;

D-21805, octadecyl-2-(trimethylarseno)-ethyl-phosphate;

Erucylphosphocholine;

Lysophosphatidylcholine;

Butylated hydroxytoluene;

Mycolactone;

Valproic acid;

Zinc undecylenate;

Phenyloin, mephenyloin, ethotoin, fosphenyloin;

Cardiac glycosides;

SAHA, Suberoylanilide hydroxamic acid;

Amphotericin B methyl ester;

Amphotericin B;

Desipramine;

Salmeterol;

Phosphatidylglycerol;

Phosphatidylcholine;

Phosphatidylethanolamine;

Phosphatidylserine;

Para-aminobenzoic acid;

Butylated hydroxyanisole;

Acetasalicylic acid;

Ceramide;

Sphingosine;

Dantrolene, 1-{[5-(4-nitrophenyl)-2-furyl]methylideneamino}imidazolidine-2,4-dione;

Tetracycline antibiotics: Tetracycline, Chlortetracycline, Oxytetracycline, Demeclocycline, Doxycycline, Lymecycline, Meclocycline, Methacycline, Minocycline, Rolitetracycline;

Phenacitin;

Sodiumdodecyl sulfate and related detergent lipid sulfate esters such as lauryl sulfate and their esters and salts;

gamma-aminobutyric acid;

4-phehylbutyrate;

Butyric acid and its esters and salts;

All short chain alkyl carboxylic acids up to chain lengths of 18 carbon atoms, their esters and salts;

Hydroximic acids such as trichostatin A;

Cyclic tetrapeptides (such as trapoxin B), and the depsipeptides;

Benzamide drugs such as Ethenzamide, Salicylamid, Alizapride, Bromopride, Cinitapride, Cisapride, Clebopride, Dazopride, Domperidone, Itopride, Metoclopramide, Mosapride, Prucalopride, Renzapride, Trimethobenzamide, Zacopride, Amisulpride, Nemonapride, Remoxipride, Sulpiride, Sultopride, Tiapride, Entinostat, Eticlopride, Mocetinostat, Raclopride, Procarbazine;

Paracetamol; and

Acetaminophen.

Examples of Secondary MAIMs include:

Cyclodextrin—depletion of lipid raft cholesterol;

Chylomicrons;

Very-low density lipoprotein VLDL;

Intermediate density lipoprotein IDL;

Low density lipoprotein LDL;

High density lipoprotein HDL;

Examples of Tertiary MAIMs include:

Cholestyramine—prevention of cholesterol uptake;

Statins—interference with cholesterol synthesis;

Folinic acids and derivatives.

When used in the sense of modulating intermolecular interactions, AAEs are classified as MMIMs. This class of molecules, because of their physicochemical properties, alters the activity of receptors made up of multiple non-covalently associated subunits. This modulatory action by the MMIM can influence the interactions of biological molecules both in the various membranes of the cell or in solution or between molecules in solution and those bound to or associated with a membrane.

AAEs are also capable of modifying the interactions of proteins that function as part of the immune system that are embedded in or associated with biological membranes. In this sense, the AAEs are classified as MAIMs. This modulatory action by the MMIM can influence the interactions of biological molecules both in the various membranes of the cell or in solution or between molecules in solution and those bound to or associated with a membrane. Many molecules, including the AAEs, are both MMIMs and MAIMs.

Through the analysis of drug effects on a variety of biological systems, it has been discovered that AAEs, in the sense that they are MMIMs, exert their pharmacological effects by changing the way in which biological macromolecules interact with each other. The MMIMs appear to act by binding to or inhibiting or in some other way reducing the ability of signaling molecules to bind to and/or activate their coordinate receptors. MMIMs and AAEs also act as MAIMs by inhibiting the formation of active dimeric Toll-like receptors (TLRs) as discussed below. In addition, it has been found that MMIMs including AAEs can inhibit the toxic activities of bacterial toxins that are composed of multiple subunits, including the toxin molecules produced by the bacterium that causes the disease anthrax. The abilities of MMIMs generally and AAEs specifically to alter the intermolecular interactions of biological molecules make them ideally suited to the treatment of a broad variety of diseases.

It has been found that the AAEs and MAIMs modify the ability of multimeric transmembrane receptors to come together to form active receptors. MAIMs and AAEs alter membrane fluidity, prevent the formation of functional receptors within membrane domains known as lipid rafts and further they prevent the assembly of multimeric bacterial toxins on cell membranes thus inhibiting the toxicity caused by the toxins. MMIMs and AAEs also diminish intermolecular interactions between free macromolecules in solution. It has also been shown that functioning as MAIMs the AAEs change membrane characteristics in a way that decreases the assembly of functional macromolecular complexes. The relative contributions of these two mechanisms of action are not known, but observations show that both effects occur.

Individual AAEs each have distinct and unique abilities to modulate intermolecular interactions occurring and consequently alter patterns of cellular physiology in solution and in lipid membranes. Taken together these observations show that the AAEs and their rationally chosen combinations may be used to treat many diseases through their ability to modulate intermolecular interactions between endogenous molecules, through the modulation of interactions between exogenous and endogenous molecules, and by modulating the interactions between exogenous molecules.

The methods of modulation include:
modification of protein-protein interactions in solution, in vesicles, in organelles and in, on or through or across membranes, either naturally occurring or man-made;
modification of protein-small molecule interactions in solution, in vesicles, in organelles and in, on or through or across membranes;
modification of protein-macromolecule interactions in solution, in vesicles, in organelles and in, on or through or across membranes;
modification of receptor-ligand interactions in solution, in vesicles, in organelles and in, on or through or across membranes;
modification of receptor mediated signal transduction;
modification of toxin-protein interactions in solution, in vesicles, in organelles and in, on or through or across membranes;
modification of the activity of endogenous receptors that are associated with membrane micro-domains such as lipid rafts;
modification of the activity of exogenous molecular species that are associated with membrane micro-domains such as lipid rafts;
modification of the activity or association of exogenous molecular species with one or more endogenous molecular species;
modification of the activity or association of exogenous molecular species with one or more endogenous species associated with membrane micro-domains such as lipid rafts;
modification of trans-membrane signal transduction;
modification of intercellular signal transduction;
modification of intracellular signal transduction;
modification of immune signaling;
modification of exocrine signaling;
modification of apocrine signaling;
modification of holocrine signaling;
modification of merocrine signaling;
modification of endocrine signaling;
modification of paracrine signaling;
modification of autocrine signaling;
modification of juxtacrine signaling;
modification of cytokine production, receptor binding, release or action;
modification of adipokine production, receptor binding, release or action;
modification of growth factor production, receptor binding, release or action;
modification of chemokine production, receptor binding, release or action;
modification of Toll-like receptor activity, ligand binding or signaling;
modification of NOD receptor activity, ligand binding or signaling;
modification of dectin receptor activity, ligand binding or signaling;
modification of G protein and G-protein coupled receptor ligand binding, activity or signaling;
modification of Notch signaling;
modification of ion channel and ion receptor activity or signaling such as calcium channels;
modification of the activity, ligand binding or signaling of receptors functional in immune signal transduction;
modification of lipid receptor activity, ligand binding or signaling;
modification of endocytosis;
modification of clathrin mediated endocytosis;
modification of caveolae formation and function;
modification of macropinocytosis;
modification of phagocytosis;
modification of exocytosis;
modification of emperopolesis;
modification of vesicle trafficking;
modification of vesicle tethering;
modification of vesicle docking;
modification of modulation of vesicle priming;
modification of vesicle fusion;
modification of the activity of SNARE proteins;
modification of neural activity;
modification of neurotransmitter receptor activity, ligand binding or signaling;
modification of endosomal acidification;
modification of membrane fusion;
modification of inter-bilayer membrane fusion;
modification of inter-cellular adhesion;
modification of membrane polarity;
modification of the activity of flippases;
modification of the activity of scramblases;
modification of the interactions of the plasma membrane and the cytoskeleton;
modification of the activity or function of caveolae;
modification of the activity or function of the glycocalyx;
modification of the activity or function of integral membrane proteins;
modification of the activity or function of lipid anchored proteins;
modification of the activity or function of peripheral membrane proteins;
modification of membrane fluidity;
modification of lipid raft structure and or function;
modification of the activity, structure or functions or proteins associated with the membrane;
modification of the activity, structure or functions of proteins associated with lipid rafts;
modification of the influence of cholesterol on biological membranes;
modification of the influence of sphingomyelin on biological membranes;
modification of the influence of sphingolipids on biological membranes;
modification of the activity, structure or function of Fc-epsilon receptors;
modification of the activity, structure or function of T cell antigen receptors;
modification of the activity, structure or function of B cell antigen receptors;
modification of the activity, structure, function or assembly of polypeptide toxins;
modification of the activity, structure or function of toxin receptors;
modification of quaternary protein structure and interactions;
modification of quaternary interactions of integral membrane proteins;
modification of quaternary protein structure and quaternary interactions of peripheral membrane proteins;
modification of the quaternary protein structure and quaternary interactions of trans-membrane proteins;
modification of tertiary protein structure of integral membrane proteins;
modification of tertiary protein structure of peripheral membrane proteins;
modification of the tertiary protein structure of trans-membrane proteins;
modification of secondary protein structure of integral membrane proteins;
modification of secondary protein structure of peripheral membrane proteins;
modification of the secondary protein structure of trans-membrane proteins;
modification of the interactions, structures or functions biological molecules that play roles in cell-cell adhesion;
modification of the activity, function or structure of proteins having beta-barrel or beta-pleated sheet structural motifs;
modification of the activity, function or structure of proteins having alpha helix structural motifs;
modification of the activity, function or structure of uniporters;
modification of the activity, function or structure or symporters;
modification of the activity, function or structure or antiporters;
modification of the activity, function or structure of voltage gated ion channels;
modification of the activity, function or structure of large conductance mechanosensitive channels;
modification of the activity, function or structure of small conductance mechanosensitive channels;
modification of the activity, function or structure of CorA metal ion transporters;
modification of the activity, function or structure of aquaporins;
modification of the activity, function or structure of chloride channels;
modification of the activity, function or structure of outer membrane auxiliary proteins;
modification of the activity, function or structure of cytochrome P450 oxidases;
modification of the activity, function or structure of OmpA like transmembrane proteins;
modification of the activity, function or structure of virulence related outer membrane protein family proteins;
modification of the activity, function or structure of bacterial porins;
modification of the activity, function or structure of complement proteins;
modification of the activity, function or structure of mitochondrial carrier proteins;
modification of the activity, function or structure of ABC transporters;
modification of the activity, function or structure of multidrug resistance transporters;
modification of the structure, function or activity of pathogen associated molecular pattern receptors;
disruption of the activity, function or structure of exogenous toxins;
disruption of the activity, function or structure of bacterial toxins;
disruption of the activity, function or structure of viral toxins;
disruption of the activity, function or structure of fungal toxins;
disruption of the activity, function or structure of chemical toxins;
disruption of the activity, function or structure of environmental toxins;
disruption or modification of the intermolecular interactions that constitute the various processes of viral capsid assembly, processing, endocytosis, exocytosis or budding;
disruption or modification of viral particle binding to cellular receptors or docking molecules;
disruption or modification of the intermolecular interactions that constitute the various processes of viral particle assembly;
disruption or modification of the intermolecular interactions that constitute the various processes of viral cholesterol homeostasis, use, processing or incorporation;
disruption or modification of the intermolecular interactions that constitute the various processes of viral particle cellular or nuclear membrane penetration;
disruption or modification of the intermolecular interactions that constitute the various processes of viral particle penetration of endocytic or pinocytic membranes;
disruption or modification of the intermolecular interactions that constitute the various processes of virus induced cellular signaling responses;
disruption or modification of the intermolecular interactions that constitute the various processes of prion interactions with endogenous targets;
disruption or modification of the intermolecular interactions that constitute the various processes of microRNA interactions with its targets;
disruption or modification of the intermolecular interactions that constitute the various processes of single and double stranded DNA interactions with its targets;
disruption or modification of the intermolecular interactions that constitute the various processes of single and double stranded RNA interactions with its targets.

Diseases that can be treated by using AAEs to modulate communications carried out or effected by biological molecules include HIV disease related cytokine-mediated neuropathy, malaria induced cytokine mediated neuropathy and tissue damage, influenza virus induced cytokine mediated neuropathy and tissue damage, bacterial infection induced cytokine mediated neuropathy and tissue damage, fungal infection induced cytokine mediated neuropathy and tissue damage, chemotherapy associated neuropathy, chemotherapy hypercytokinemia associated dementia, amelioration of hypercytokinemia induced HIV disease related dementia, diseases involving an organism making use of or that stimulates the host immune system to produce or release cytokines, chemokines, growth factors or other signaling molecules as part of the phathophysiology of the disease, diseases involving an organism for which cholesterol is an essential nutrient, virulence factor or host factor, cancer, cancer associated cachexia, cholera, Buruli ulcer, anthrax, Staphylococcal enteritis, acne, rosacea, Tinea spp. infections, influenza, Neisseria meningitidis infections, meningitis, Helicobacter infections, HIV 1 infection, HSV 1 infection, HSV 2 infection, HPV infection, chlamydia, gonorrhea, syphilis, trypanosome infections, malaria, kinetoplastid infections, yeast infections, Cryptococcus infections, Candida infections, hepatitis A virus infection, hepatitis B virus infection, hepatitis C virus infection, bacterial meningitis, viral meningitis, fungal meningitis, Leishmania infections, filovirus infections, Ebola virus infections, Marburg virus infections, tuberculosis, leprosy, Mycobacterium marinum infections, bilharzia, schistosomiasis, Schistosoma mansoni infections, Schistosoma haematobium infections, Schistosoma japonicum infections, Yersinia infections, Yersinia pestis infection, shigelosis, Clostridium perfringens infection, Vibrio cholerae infection, Systemic inflammatory response syndrome, sepsis, cytokine storm or hypercytokinemia, multiple organ dysfunction syndrome, graft versus host disease, acute respiratory distress syndrome, avian influenza, smallpox, disseminated intravascular coagulation, catastrophic antiphospholipid syndrome, antiphospholipid syndrome, multiple organ dysfunction syndrome, Stevens Johns syndrome, toxic epidermal necrolysis, pemphigus, psoriasis, systemic sclerosis, systemic lupus erythematosis, multiple sclerosis, Crohn's disease, inflammatory bowel disease, diabetes type 1, diabetes type 2, diabetes of pregnancy, arteriosclerosis, atherosclerosis, arteriolosclerosis, hypertension, seasonal allergy, delayed type hypersensitivity, contact allergy, alcoholic hepatitis, non alcoholic fatty liver disease, vitiligo, rheumatoid arthritis, osteoarthritis, eosinophilia, acute and chronic nephritis, post surgical neuropathy, ischemia-reperfusion injury, stroke, ischemia, systemic inflammatory disorder, endometriosis, pelvic inflammatory disease, sterile meningitis, carpal tunnel syndrome, chronic fatigue syndrome, Gulf war syndrome, compartment syndrome, pancreatitis, inflammatory bowel disease, gastroesophageal reflux disease, colitis, hemorrhoids, osteoarthritis, traumatic brain injury, brain hemorrhage, rhabdomyolysis, septic shock, toxic shock syndrome, idiopathic pulmonary fibrosis, mesothelioma, brown lung, injuries and irritations of the lung due to the presence of irritating particles, fibers and dusts, and bursitis.

What will be described herein is the composition of the AAEs, Formula I—R₂OOC—(CH₂)ₙ—COOR₁, as well as example experiments showing the operation and effectiveness of the AAEs.

The mixtures of azelaic acid ester derivatives of the present invention are certain esters that show a higher lipophilicity and biphase solubility than the parent compound and hence are better able to be incorporated into a pharmaceutical formulation.

Examples of suitable straight-chain alkyl groups (R1 and R2) in Formula I include methyl, ethyl, propyl, butyl, pentyl, hexyl, heptyl, octyl, dodecyl, palmityl, stearyl and the like groups.

Examples of suitable branched chain alkyl groups include isopropyl, sec-butyl, t-butyl, 2-methylbutyl, 2-pentyl, 3-pentyl and the like groups.

Examples of suitable cyclic alkyl groups include cyclopropyl, cyclobutyl, cyclopentyl and cyclohexyl groups.

Examples of suitable “alkenyl” groups include vinyl (ethenyl), 1-propenyl, i-butenyl, pentenyl, hexenyl, n-decenyl and c-pentenyl and the like.

The groups may be substituted, generally with 1 or 2 substituents, wherein the substituents are independently selected from halo, hydroxy, alkoxy, amino, mono- and dialkylamino, nitro, carboxyl, alkoxycarbonyl, and cyano groups.

The expression “phenalkyl groups wherein the alkyl moiety contains 1 to 3 or more carbon atoms” means benzyl, phenethyl and phenylpropyl groups wherein the phenyl moiety may be substituted. When substituted, the phenyl moiety of the phenalkyl group may contain independently from 1 to 3 or more alkyl, hydroxy, alkoxy, halo, amino, mono- and dialkylamino, nitro, carboxyl, alkoxycarbonyl and cyano groups.

Examples of suitable “heteroaryl” are pyridinyl, thienyl or imidazolyl.

As noted herein, the expression “halo” is meant in the conventional sense to include F, Cl, Br, and I.

Also included are all molecules of the aforementioned types including substitutions of 1 or more deuterium atoms in the place of one or more hydrogen atoms. Such substituted molecules are well known in the art to possess different pharmacological and pharmacodynamic properties relative to those of the un-substituted molecules that will give rise to therapeutic advantages such as longer biological half life, altered receptor affinity and other such effects encompassed within the realm of metabolic differences due to heavy isotope effects.

Among the compounds represented by the general Formula I, preferred compounds are such in which R1 and R2 are the same and is one of the following groups:

Methyl, ethyl, n-propyl, iso-propyl, n-butyl, iso-butyl, sec-butyl, t-butyl, n-pentyl, 2-pentyl, 3-pentyl, sec-pentyl, iso-pentyl, neo-pentyl, n-hexyl, 2-hexyl, 3-hexyl, sec-hexyl, iso-hexyl, cyclohexyl, palmityl, stearyl, methoxyethyl, ethoxyethyl, benzyl and or nicotinyl.

Among the compounds represented by the general Formula I, preferred compounds are such in which R1 and R2 are the different and is one of the following groups:

Methyl, ethyl, n-propyl, iso-propyl, n-butyl, iso-butyl, sec-butyl, t-butyl, n-pentyl, 2-pentyl, 3-pentyl, sec-pentyl, iso-pentyl, neo-pentyl, n-hexyl, 2-hexyl, 3-hexyl, sec-hexyl, iso-hexyl, cyclo-hexyl, palmityl, stearyl, methoxyethyl, ethoxyethyl, benzyl and or nicotinyl.

And the other, R2, is also taken from the above list but is not the same as R1.

Other preferred compounds are such in which R1 is hydrogen and R2 is one of the groups listed above, or R2 is hydrogen and R1 is one of these groups.

The compounds of Formula I are esters (mono and di-esters) of azelaic acid formed either at C1 or C9, or at both C1 and C9 carboxyl groups. Several esters of dicarboxylic acids have long been known and the information on the preparation or pharmacological activity of various esters of dicarboxylic acids can thus be found in the cited references. However, these references or other information in the literature do not disclose or indicate the esters of azelaic acid and any utility of esters or other derivatives of azelaic acid as drugs suitable for oral, vaginal, rectal, parenteral, intravenous, intrathecal, ocular, sub-cutaneous, intramuscular, trans-dermal, trans-epithelial, trans-mucosal, by inhalation or insufflation, and topical delivery of AAEs, nor any properties of the compounds that might indicate such utility.

The compounds of Formula I can be prepared by various methods as already described in the literature for a number of AAEs (see the references cited above). A large number of methods are known to the art that will allow a skilled practitioner to produce the claimed composition of matter or its analogs and homologues. Among these are for instance: The direct formation of the ester from the requisite acid and an alcohol. This condensation may be achieved by the dehydration of the reaction mixture with a suitable agent or by heating a mixture of the acid and alcohol. Commonly used dehydrating agents and methods include, heat, concentrated acids such as sulfuric acid, acid anhydrides such as phosphorous pentoxide, gaseous acids such as hydrogen chloride gas introduced into a solution of the acid in the requisite alcohol, solution chemistries formed by reaction mixtures such as iodine or bromine with sodium hypophosphite or red phosphorous that generate hydriodic acid in-situ which then goes on to promote the formation of the ester by dehydration or transient organohalide formation, and so on. This listing should not be taken as being all-inclusive or exhaustive for there are many additional dehydration mediated esterification methods are known to the art.

A second major set of synthetic strategies comprise the methods wherein an activated intermediate of either the acid or the alcohol is formed which is then further reacted with the appropriate esterifying alcohol or acid to produce the desired ester. Among these are reactions of an alcohol with an activated form of the acid. Activated forms of the acid include acid halides, acid anhydrides including both homo- and hetero-anhydrides, the reaction of the internal anhydride of the parent acid with the requisite alcohol, esters and anhydrides of both the acid and the alcohol which are formed by reaction of the requisite acid or alcohol with p-toluene sulfonyl chloride to produce the tosyl anhydride or ester which is subsequently reacted with the alcohol or acid respectively to produce the desired final ester. Similarly one could substitute a simple organic acid anhydride, such as acetic acid anhydride, for the p-toluene sulfonyl chloride. In addition one could start with one ester selected from among the desired compositions of matter and by the means of solution of the ester in a desired alcohol in the presence of an appropriate acidic or basic catalyst effect a conversion of the starting ester of the acid to an ester wherein the alcohol becomes that in which the reaction is carried out which method is also known to the art as trans-esterification.

For example, one could start with the dimethyl ester of the acid and by solution of the dimethyl ester in ethanol in the presence of an acid or base one could cause the facile formation of the diethyl ester of the acid. In addition, if a mixed ester of the acid were desired, one could utilize an appropriately composed solution of the two desired alcohols in any of the methods herein described.

One could resort to the use of halogenated intermediates or ingredients to form the required esters. For example, thionyl chloride will chlorinate both acids and alcohols, thereby resulting in the acyl and alkyl chlorides. These acyl and alkyl chlorides may then be further reacted with the desired alcohol or acid respectively to produce the desired ester products. Other common halogenating agents include for example oxalyl chloride and the chlorides and bromides of phosphorous such as phosphorous penta- or trichloride and penta- or tribromide or phosphorous oxychloride.

Finally, it is commonly practiced to form esters through the action of a strong base on a mixture of the acid and the alcohol. Examples of strong bases include lithium aluminum hydride and other metal hydrides, alkali metal alkoxides such as sodium ethoxide and diisobutyl aluminum hydride, sodium or potassium hydroxide, sodium or potassium peroxide and so on.

This listing of materials and methods should not be interpreted to be limiting, exhaustive or all-inclusive but is merely presented for illustration of the claimed possible methods. In addition, any of the above methods may be used with appropriate modifications of the reactants and conditions to produce mono-esters of the diacid, homo-diesters of the diacid or hetero-diesters of the diacid.

As mentioned above, this invention is generally directed to esters of azelaic acid. Such AAEs, when administered to a warm-blooded animal in need thereof, have utility in the prevention or treatment of conditions enumerated above in warm-blooded animals, including humans.

It has been found that the esters of azelaic acid have good and beneficial characteristics that are such as to render them particularly suitable for use in pharmaceutical formulations. Owing to the simple conception and low cost of the present invention, the procedures described in this invention easily lend themselves to the adaptation of the preparation methods on an industrial scale.

The examples given illustrate how of azelaic acid esters may be used, as well as prove their effectiveness. Only a few of the many possible embodiments that may be anticipated are shown by these examples, which are intended to define, in a non-limiting sense, the scope encompassed by the invention.

The present invention and its research show that various cells and tissues of the body communicate with each other using a variety of means, including the transmission of electrical impulses and by producing and releasing various small and large molecules, such as proteins. This communication between cells is essential to maintaining the structure and function of the concerned cells and tissues and ultimately the integrity of the whole organism.

The brain, for example, produces and receives electrical impulses through the afferent and efferent nervous systems. Neurotransmitters such as acetylcholine, epinephrine (adrenaline), and dopamine are synthesized and released by nerve cells as media of communication with both other nerve cells and the tissues of the body. Protein signaling molecules such as insulin, leptin, and the cytokines, chemokines and growth factors all interact with receptors on the cells of the nervous system and further with cells of the entire organism.

Thus, this chemical communication is essential to maintaining the organism. Every cell in the body engages in biomolecular communication. Another vital role of this communication network is the mounting of effective preservative responses to infection, illness and injury. The immune system is composed of a variety of tissues and specialized types of cells that operate as a coordinated network in a complicated and incompletely understood fashion, the purpose of which is to respond effectively to various physiological challenges.

The immune system can roughly be divided into two interdependent functional components, the innate immune system and the adaptive immune system.

The various components of the immune system exchange information in order to function. These communications are affected by direct cell-to-cell contacts and through the actions of soluble signaling molecules. One example of cell-to-cell communication is the interaction between antigen presenting cells and effector cells. For example macrophages and T cells communicate in this face-to-face fashion. Soluble signaling molecules include many different types of proteins and non-protein small molecules. Some examples of proteinaceous signaling molecules are the hormones, interleukins, chemokines, cytokines, etc. Small molecule signals include prostaglandins, leukotrienes and neurotransmitters such as epinephrine.

For the purposes of this discussion and to avoid unnecessary complexity these soluble protein mediators will be referred to as ‘cytokines’.

Derangements and dysfunctions of the immune system underlie the pathophysiology of many diseases. Excessive, unbalanced or inappropriate immune system responses have been found to play important roles in cancer, autoimmune diseases, allergies and the so-called ‘hypercytokinemias’ such as septic shock and malaria.

Cancers recruit various kinds of immune cells to migrate into the tumor mass where they are ‘enslaved’ by the tumor in furtherance of its survival and growth. The mechanisms by which the cancer induces the cooperation of immune system cells all involve communications through soluble mediators such as those discussed above. Control of this communication has shown promise for the treatment of various cancers.

Medical science has recognized the need to control this communication in order to treat certain diseases. A variety of strategies have been employed to modify this communication including antibodies that bind to and deactivate various immune signaling molecules or their receptors and soluble receptor analogs that bind to signaling molecules and prevent them from reaching their destination receptors on cells. In addition, the signaling molecules themselves have been used as therapies. For instance the cytokine interleukin-2 is used for the treatment of various cancers and chronic viral infections. These types of drugs are known as ‘targeted’ therapies because of their high specificity.

Targeted therapies using synthetic antibodies designed to bind to and deactivate various cytokines are currently used in medical practice. For example, the drugs Remicade and Humira are synthetic antibodies that bind to and deactivate the cytokine tumor necrosis factor (TNF). These drugs are used to treat various autoimmune diseases such as psoriasis, Crohn's disease and rheumatoid arthritis. The drug Actemra, also an antibody, binds to and deactivates the receptor for the cytokine interleukin-6 and is used for the treatment of rheumatoid arthritis and Castleman's disease.

In addition, other drugs are known to influence intra- and intercellular signaling pathways. Some examples include the non-steroidal anti-inflammatory drugs (NSAIDS) such as aspirin. Most NSAIDS exert therapeutic anti-inflammatory effects though the inhibition of the COX enzymes. COX enzymes make the pro-inflammatory prostaglandins and thromboxanes. However, there are additional biological effects of various NSAIDS that cannot be explained solely by their COX inhibiting activity.

In all of these types of communication there are at least two distinct molecules that are involved; the signaling molecule, also known as the ‘ligand’, and its receptor or receptors. TNF for example binds to the TNF receptor (TNFr).

When a signaling molecule binds to its receptor on the surface of or within a cell, it activates that receptor to pass the information to the cell. The cell then responds to the signal. The response may take any of a number of forms including the initiation of DNA synthesis in preparation for division, the release of signals into the extracellular milieu, or the cell may die by initiating programmed cell death, also known as apoptosis. These are only a few examples of the possible responses of a cell to receipt of a signal.

Many receptors are composed of two or more non-covalently bonded subunits. These subunits must come into physical proximity in order to form a functional receptor.

One example of these types of multi-subunit receptors are the Toll Like Receptors (TLRs). TLRs form dimers, composed of receptor two subunits, which sense various molecules associated with pathogens. TLRs are currently thought to be active as receptors only in the dimer form.

There are many other types of receptors present on the surface of and inside mammalian cells that are composed of multiple subunits.

Many types of receptors are attached to or embedded in the plasma membrane of the cell, which is the outer perimeter of the cell. Many receptors have a structure that can be divided into three regions; a part that is outside the cell, a part that penetrates through the plasma membrane, and a part on the inside of the cell. These regions are referred to as the ‘extracellular’, ‘transmembrane’ and ‘intracellular’ domains respectively. It is these domains that interact with those on other subunits to form active receptors.

The plasma membrane forms a physical barrier which contains the cytoplasm and nucleus and various organelles. The plasma membrane serves as a border that physically separates interior of the cell from the outside world. The plasma membrane is composed of lipids, proteins, polysaccharides and their compounds. Some examples of membrane lipids include phosphatidylcholine, phospatidylethanolamine and phosphatidylserine and cholesterol. Many of these lipids have additional bound species such as proteins (lipoproteins) and complex polysaccharides (membrane bound polysaccharides).

The plasma membrane with its integral and peripheral components is an extremely dynamic structure.

Protein receptors and some membrane lipids are thought to cluster together in specialized regions or “islets” known as lipid rafts. The lipid rafts are more organized and tightly packed than the surrounding bilayer, but float freely in the membrane bilayer.

Lipid rafts serve as organizing centers for the assembly of receptors and signaling molecules, influencing membrane fluidity and membrane protein trafficking.

On and within the plasma membrane are a variety of receptors that serve as sensors for the presence of pathogens which serve as a ‘border patrol’ allowing the cell to respond appropriately to infection and injury.

Given the universality of biomolecular communication, the diversity of molecules and receptors involved in the communication and the central role that disordered or inappropriate communication plays in many diseases, it is clear that there is a great need for medicines that can alter the signal traffic.

As discussed above many medicines currently in use are either narrowly targeted to act on a specific single signaling pathway, or are broadly acting with several recognized mechanisms of action and minor ‘off-target’ activities.

In summary, all cellular communications and signaling depend on some sort of intermolecular interactions between various biological molecules. Under normal conditions, this communication assures an appropriate response of cells and the entire organism to ever changing conditions and environmental challenges. Under pathological conditions exemplified by pathogen infections this communication can be corrupted and can in fact be subverted and used by the pathogen to exploit the host immune response for own benefit. Interference with the pathogen communication via modulation of key macromolecular interactions is one way that has been used to allow the immune system to restore a normal, healthy state.

Diseases such as psoriasis, diabetes, rheumatoid arthritis, scleroderma, lupus, Crohn's disease, amylelotrophic lateral sclerosis (ALS), multiple sclerosis (MS), and others raise the question of the origin or initiating factor of the autoimmune response that causes or precipitates the self-destructive malfunctions of the immune system and its various components. It is documented that many of these diseases are mediated by and through the actions of autoreactive cell mediated immune responses. The current methods of palliating these diseases revolve around inhibiting the autoimmune response by the administration of immunotoxic drugs such as cyclophosphamide, methotrexate or more recently by treatment with biological response modifiers that, for example, inhibit TNF function. TNF (cachexin or cachectin and formally known as tumor necrosis factor-alpha) is a cytokine involved in systemic inflammation and is a member of a group of cytokines that stimulate the acute phase reaction. All of these treatments are administered based on the premise that the immune system has become self-reactive and/or overactive with a resulting detriment to the patient suffering from the condition and that it is the immune system or a component of the immune system that is primarily defective.

Paradoxical though is the observation that in physiological states where the immune system activity is diminished, such as in patients undergoing antineoplastic chemotherapy or radiotherapy, both of which diminish immune response, these autoimmune diseases are often induced or become clinically apparent. This phenomenon is also observed in diabetics who go on to develop psoriasis or rheumatoid arthritis notwithstanding the decrement in their immune response brought on by their hyperglycemic physiological status.

These observations raise a very important question: If the immune system is responsible for the autoimmune diseases, why does decreasing the activity of the immune system often provoke the development of many of these same autoimmune diseases?

It is clear from the accumulated scientific evidence that autoimmune diseases arise out of imbalances in the functions between the various components of the immune system. The disordered patterns of signaling molecule expression and release are more than simple symptoms; they are key mechanisms in the pathophysiology of these diseases.

The AAEs and various mixtures of AAEs are capable of broadly modulating inter- and intra-cellular signaling and as such they have great utility in the treatment of diseases and other conditions where that signaling is deranged or plays a role in the pathophysiology of the disease. A discussion of all of these disorders would be quite lengthy but is shown by several experimental examples that detail the use of AAEs and illustrate their mechanism of action.

### Example 1

In the first example, it is demonstrated that contrary to what classical pharmacological inference would suggest, each of the different AAEs has biochemical effects that are considerably different from the other homologous AAEs. Using the MatTek EpiDerm™ in vitro human skin model system, EpiDerm tissues were exposed to the plant derived irritant croton oil. Tissues were also exposed to the various AAEs. After 24 hours of exposure to the various irritant/AAE treatments, the tissues and their supporting growth media were removed for analysis by multiplex immunoassay. The markers measured represent a range of cytokines, chemokines, growth factors and signaling molecules known to be of significance in intracellular and intercellular communication and regulation. In addition many of these markers are known to play key roles in a variety of diseases.

The results of the experiment indicate clearly that each of the AAEs possesses pharmacological activity. In addition, each of the AAEs showed a pattern of activity that was different from the other AAEs tested.

The results showed that the AAEs all possess different pharmacological activities in this model system. For instance, dimethylazelate (DMA) induced increases in medium marker levels, or up-regulation, of a number of measured markers. Of note, some of these markers are known to have anti-inflammatory properties (e.g. IL-4 and IL-10) and it also increased production of pro-inflammatory makers such as IL-1-beta and TNF-alpha. In contrast, in the tissue measurements for DMA these same markers were decreased relative to control.

One possible interpretation of these results is that DMA suppresses local inflammation while promoting simultaneously inflammatory and anti-inflammatory longer-range signal production.

The data obtained for specimens treated with diethyl azelate (DEA) clearly showed a pattern of marker modulation was distinctly different from that observed in the case of DMA.

The other AAEs in the treated series likewise show unique patterns of signal modulation.

Minor alterations in molecular structure, such as those between ethyl and methyl esters, are usually presumed to induce corresponding minor changes in biochemical activity. Our results however demonstrate significant differences of activity for DMA and DEA in contradiction to the received pharmacological wisdom.

Further, the data demonstrate that by making rational choices directed toward achieving desired patterns of signal modulation, one can use these data to select various esters for use together to achieve pharmacological results tailored to the specific disease or condition being treated. For example, combining DMA with DEA will produce a product that simultaneously increases the anti-inflammatory cytokines IL-4 and IL-10 (due to DMA) while suppressing the pro-inflammatory cytokines IL-17, IL-8 and IL-23 (due to DEA).

Thus the AAEs each have different pharmacological properties that can be used in combination to treat a broad range of diseases associated with derangements of cellular signaling.

Using these data, a lead drug, HF1107 has been developed by selecting from among the various esters having complementary activities with the objective of attaining desired therapeutic endpoints in a number of model systems.

### Example 2

The second example experiment shows that although pharmacologists and drug designers, as well as the prior art, consider esters to be pro-drugs that break down after administration to release the active drug, which then exerts the desired therapeutic effects, this is not an important factor in the pharmacology of the AAEs. In an experiment analogous to the one discussed above, the effects of the AAEs were compared to the parent compound azelaic acid.

As described above, EpiDerm tissues were treated with croton oil irritant and/or with counter irritant treatments. Differential cytokine responses were measured by multiplex immunoassay and results were expressed relative to control, i.e. tissues exposed only to croton oil.

In the case of IL-17, the data indicate that tissue levels of IL-17 in DEA treated tissues were exceedingly high relative to control, while those for the tissues treated with buffered azelaic acid were significantly lower than control. A similar pattern of opposing drug induced differential responses is evident for IL-2, MCP-1, RANTES, ENA-78 and so on. On the other hand, for the marker MIP-1-alpha tissue levels of DEA and buffered azelaic acid were both elevated relative to control.

A pattern of opposing and parallel differential responses is also evident in the corresponding measurements made in the growth medium of the samples.

Taken together these data clearly demonstrate that, while they are in some ways similar, the differences in activity observed between DEA and azelaic acid are so large that it is evident that they are truly different drugs.

While the AAEs are metabolized to azelaic acid and the corresponding alcohols over time, these data show that, on the pharmacologically relevant time scale, the biochemical actions attributable to azelaic acid are minor relative to those of the esters.

### Example 3

This experiment demonstrated that while much of the scientific literature, medical literature and prior art emphasizes the antibacterial activity of AAEs is primarily exerted through direct killing of bacteria by damaging the bacteria the AAEs have important biological activities at concentrations well below those that have antibacterial activity. To investigate this area a number of experiments were conducted using some pathogens that infect the skin.

The antibacterial activity of AAEs were evaluated by an in vitro antibacterial activity assay, in which Staphylococcus aureus bacteria growing in culture were exposed to various concentrations of HF1107. The number of live bacteria was estimated by measuring the absorbance of the medium containing the growing bacteria at various times after drug exposure. Increases in absorbance correlate with increases in the number of bacteria, and decreases in absorbance correlate with decreases in the number of bacteria. No change in absorbance indicates that the bacteria were not multiplying, but did not necessarily indicate that they were dying.

Using 12.5% HF1107, it was observed that the absorbance over time decreased. For 0% HF1107, absorption increased over time. When 3.12% HF1107 (absorbance trending down with time) was compared to those at 1.58% HF1107 (absorbance trending up with time) it was clear that the bacteria were prevented from growing at some concentration between these two concentrations of HF1107.

Similar responses were observed in studies with the organism Mycobacterium ulcerans. The absence of apparent bacterial growth at 5% HF1107 concentration, when compared to that treated with 1% HF1107 indicates that M. ulcerans growth is inhibited at some concentration between one and five percent HF1107.

These results, as detailed in example experiments 1 and 2 above, and in example experiment 4 below, indicate that while the AAEs have antibacterial properties, these effects are observed at relatively high concentrations, i.e. in the percent by weight concentration range.

At non-antibacterial concentrations it can be shown that the AAEs have a demonstrable effect on the immune system and the cells and tissues of the body.

The immune system is equipped with a number of mechanisms by which it defends and maintains the integrity of the body. Vital among these is the detection and destruction of biological invaders such as bacteria, fungi and viruses.

A number of general immune responses to invaders have been characterized and these responses can be roughly divided into two categories. The first of these is the innate immune system, the second is the adaptive immune system.

The innate immune system is generally considered to be composed of various sets of cells that function together to mount a primary cell mediated attack on invaders. The adaptive immune system is also composed of classes of cells which also function to respond to and attack invaders, but in addition the adaptive immune system can ‘remember’ past attacks such that any future attacks by the same invader are recalled in such a way that the invader is more promptly eliminated.

In operation, the innate immune system is responsible for a prompt general immune response, and it acts immediately on sensing the presence of an invading organism while the adaptive immune system must first learn the nature of the invader before responding and killing it. The border between the innate and adaptive immune systems is indistinct as there is considerable overlap and cross-talk between the various types of cells in each system and some cell types perform roles in both systems.

Cells of the innate immune system act in many ways as sentinels, patrolling through the tissues looking for signs of infection. On detecting an invader they respond by sending signals to other types of cells and they also may attack the invader directly depending on their type.

The patrolling cells have a variety of sensors that allow them to detect invaders. These sensors are known as Pathogen Associated Molecular Pattern (PAMP) receptors. There are a number of different classes of PAMP receptors, among them are the Toll like receptors (TLR), the nucleotide oligomerization domain receptors (NOD), dectin receptors and so on.

Experiments were conducted using selected commercially available dendritic cells as a model. Dendritic cells are among the first immune cells to identify invading pathogens and they have many types of PAMP receptors that allow them to perform their surveillance functions. A variety of receptor agonists (an agonist is a substance that binds to and activates a receptor) were used to evaluate the effects of AAE treatment on TLR receptor function.

Agonists activate PAMP receptors of the dendritic cells. Activation of PAMP receptors causes cells bearing these receptors to react and one of the types of reactions is the release of various signaling molecules such as those described and measured in the foregoing examples.

### Example 4

This experiment involved the addition of various PAMP receptor agonists to dendritic cells in culture in the presence or absence of azelate esters and measuring the levels of cytokines, chemokines, growth factors and other signaling molecules that the cells released in response to these treatment conditions.

The data showed the effects of treatment with HF1107 plus a receptor agonist relative to the effect of the receptor agonist alone. One of the markers measured in the experiment was released (extracellular) adenosine triphosphate (ATP), which functions both as a molecular unit of energy but also as a type of signal of cellular distress or danger. As a danger signal, ATP has been found to play a key role in diseases such as asthma. The results clearly showed that HF1107 decreases the release of the ATP danger signal in agonist stimulated cells.

Significantly, the concentration of HF1107 used in this experiment was 0.025%, well below the lower limit of antibacterial activity observed in example experiment 3.

Cytokine data for this experiment were also acquired and showed significant decreases in quantities of a number of released cytokines.

As demonstrated in the previous examples, deviations of the data above the zero percent change level indicate increases relative to un-treated controls, and those below indicate decreases relative to controls. Notable is the similarity of the data for the various classes of TLRs, as all of the TLRs within each class responded in a similar fashion and many of the TLR types studied are localized to the outer leaf of the cellular membrane. TLRs that are not present on the plasma membrane (TLRs 7 and 8) respond differently from those that are, but the various TLRs all responded in ways that were similar to one another depending on their class.

TLRs are all composed of dimeric supramolecular structures assembled in membranes. Association of TLR receptor subunits is a necessary condition of function.

Additional experimentation, as shown in the next two examples, demonstrates that AAEs are capable of modulating interactions between biological molecules generally and are quite active in the modulation of the activity of proteins that form supramolecular assemblies in and on the plasma membrane.

Because of the apparent ability of the AAEs to modulate the intermolecular interactions of biological molecules the activities of the AAEs against various bacterial toxins was examined.

Many bacterial toxins are composed of multiple subunits. These subunits must, as part of the mechanism of action of the toxin, assemble to form noncovalent supramolecular complexes. Commonly known examples of toxins of this type include anthrax toxin produced by Bacillus anthracis, cholera toxin produced by Vibrio cholerae, and the Shiga type toxin produced by the bacterium that was in May of 2011 was responsible for the food associated outbreak of Escheria coli O104:H4 in Europe.

### Example 5

In the fifth example experiment, anthrax toxin (ATX) was examined. ATX is composed of three proteins. These are known as protective antigen (PA), lethal factor (LF) and edema factor (EF). PA is the first subunit to bind to receptors on the surface of a cell. There are two known types of receptor, TEM8 and CMG2. PA binds to these receptors and then the PA-receptor complex translocates across the surface of the cell to a lipid raft membrane microdomain. In the lipid raft the PA-receptor complexes associate with each other to form supramolecular assemblies composed of seven or eight PA-receptor complexes in a ring or circular arrangement. These supramolecular assemblies are canonically referred to as ‘heptamers’ or ‘octamers’. The LF and or EF then bind to the top of the heptamer/octamer complexes. The fully assembled ATX complex composed of seven or eight molecules of PA and one or more molecules of LF and EF, is then taken into the cell via endocytosis. Following a number of intermediate steps, the LF and EF are then injected by the PA heptamer/octamer into the cytoplasm of the cell. Once in the cytosol the LF and EF go on to damage the machinery of the cell. EF causes increases in cyclic adenosine monophosphate resulting derangement of water homeostasis. LF cleaves off a part of mitogen activated protein kinase kinase (MAPKK), a key intermediate in the inflammatory response pathway responsible for sensing pathogens that is mechanistically down stream from the TLRs discussed above. Cleavage of MAPKK by LF causes cells to lose the ability to respond to molecules that stimulate TLRs, i.e. the agonists discussed above. Thus exposure of cells to TLR agonists was used to probe the ability of the cells to mount an appropriate inflammatory response after exposure to ATX and how that response was modulated by treatment with AAEs. If the cells had been intoxicated by LF, TLR agonist treatment could not induce the production and release of inflammatory cytokines. If however the drug prevented the toxicity of LF the TLR agonist response would be preserved.

This experiment investigated the PA and LF components of ATX. In these experiments mice were exposed to a mixture of PA and LF with and without treatment with HF1107. The blood of the mice was then removed, the circulating immune cells were then exposed to the TLR agonist bacterial lipopolysaccharide (LPS), which binds to and strongly activates TLR-2/4 causing the cells to release a burst of inflammatory cytokines. As described above LF causes cells to lose the ability to release this inflammatory cytokine burst in response to LPS stimulation.

The results show that HF1107 treated mice produced more proinflammatory cytokines (IL-1alpha, IL-2, KC (mouse IL-8), MCP-1, IFN gamma, IL-6, GM-CSF) on LPS stimulation than did the mice that were not treated with the exception of the markers MIP-1-alpha, IL-1-beta RANTES and TNF-alpha. The data also show that the basal cytokine level in the plasma of the treated animals showed significant increases of the proinflammatory markers IL-1-beta, IFN-gamma and TNF-alpha relative to untreated animals. Taken together these results indicate that HF1107 prevented ATX immunotoxicity in the treated animals and the results provide support for the hypothesis that HF1107 disrupts the formation of active PA heptamers/octamers on the cell surface thereby preventing LF entry and toxicity or alternatively that HF1107 prevents functional association of LF association with the PA heptamers/octamers bound on the cell surface thereby preventing ATX toxicity.

### Example 6

In the sixth example experiment, the ability of AAEs to modulate the activity of cholera toxin was examined. Cholera toxin (CTX) is produced by the bacterium Vibrio cholerae and is responsible for the profuse watery diarrhea that characterizes the disease cholera. Cholera toxin is also a multimeric toxin, known as an AB₅ toxin. The A subunit has enzymatic activity and is an ADP ribosylase. The B subunit binds to GM1 gangliosides on the surface of the host/victim cell and forms pentameric units on the cell surface. Analogously to ATX, the A subunit then binds to a pentamer of membrane receptor bound B subunits. The entire complex then is internalized via endocytosis which takes place on a lipid raft membrane microdomain on the cell surface.

In this experiment, human peripheral blood mononuclear cells were exposed to cholera toxin B subunit. After exposure to the B subunit, the cells were treated with an antibody to which a fluorescent reporter molecule was attached. Thus if B subunit pentamers were formed in the lipid rafts of the cell the cells would appear on observation through a fluorescence microscope to have regions of high fluorescent intensity, visualized as glowing spots.

Human PBMCs were treated with HF1107 and then exposed to cholera toxin B subunit followed by exposure to fluorescently labeled anti-B-subunit antibody and compared to a control group of PBMCs, which were also exposed to cholera toxin B subunit and anti-B-subunit antibody. When microscopically visualized with fluorescent illumination, the control (untreated) group showed fluorescent pinpoints on their cell membranes, with close up views showing the localization of the labeled B subunit to lipid rafts on the cell membrane. The HF1107 treated group clearly showed no cells having fluorescently labeled B subunit clusters present on their surfaces. This indicated that the HF1107 has disrupted B subunit pentamer formation and by extension CTX function. These results strongly support the hypothesis that the AAEs disrupt macromolecular interactions and that the AAEs represent viable treatments for both cholera and anthrax.

In addition, experiments conducted with whole cell lysates of methicillin resistant Staphylococcus aureus (MRSA) and Mycobacterium ulcerans (MU) have also shown the results that parallel those obtained for cholera and anthrax toxins, i.e. the toxins produced by these bacteria were disabled and immune cell function and viability were preserved by treatment with HF1107. Increased survival in live bacteria in vivo challenge experiments and preservation of immune function in PBMCs taken from treated animals has been shown. The particular MRSA used in these experiments produced a number of exotoxins including the multimeric pore forming toxin Panton-Valentine Leukocidin. MU produces the cytotoxic/immunotoxic macrolides known as mycolactones in addition to other as yet uncharacterized membrane acting toxins. HF1107 treatment of human PBMCs preserved immune cell function in challenges with whole-cell lysates of both of these bacteria.

From these experiments and observations, examples of methods and formulations for treatment have been established:
1) Low dose intravenous or subcutaneous administration—AAEs may be formulated for intravenous administration by combination of the ester or esters with one or more amphiphilic surfactant molecules. One such surfactant is Polysorbate 80. Half percent (0.5%) by weight of AAEs is/are added to a solution of 0.1% by weight Polysorbate 80 USP in sterile water for injection USP. The solution is thoroughly mixed to ensure solubilization of the AAEs. The solution is then administered by intravenous or subcutaneous infusion as required.
2) High dose intravenous administration—AAEs may be formulated for intravenous administration by combination of the ester or esters with one or more amphiphilic carrier molecules. One such carrier molecule is human serum albumin. Up to 25% by weight of AAEs is/are added to a solution of 5% by weight human serum albumin in pH 7.4 phosphate buffered saline for injection USP. The solution is thoroughly mixed to ensure solubilization of the AAEs. The solution is then administered by intravenous infusion as required.
3) Long acting intravenous administration or intraperitoneal—AAEs may be formulated for intravenous administration where the duration of drug effect is desired to be extended in time by combination of the ester or esters with one or more amphiphilic carrier molecules having the property of slowly releasing the esters. One such carrier molecule is hydroxypropyl-beta-cyclodextrin. Up to 1% by weight of Azelaic Acid Ester(s) is/are added to a solution of 0.5% by weight hydroxypropyl-beta-cyclodextrin in pH 7.4 phosphate buffered saline for injection USP. The solution is thoroughly mixed to ensure solubilization of the AAEs. The solution is then administered by intravenous or intraperitoneal infusion as required.
4) Intrathecal or subcutaneous or parenteral administration
AAEs may be formulated for intrathecal administration where the location of drug effect is desired to be the central nervous system by combination of the ester or esters with one or more amphiphilic carrier molecules having the property of slowly releasing the esters. One such carrier molecule is polyethylene glycol having an average molecular weight of 3400 Daltons (PEG3400) 1% by weight of AAEs is/are added to a solution of 2.5% by weight PEG3400 in normal saline for injection USP. The solution is thoroughly mixed to ensure solubilization of the AAEs. The solution is then administered by intravenous or subcutaneous or intraperitoneal infusion as required.
5) Subcutaneous administration long acting—AAEs may be formulated for subcutaneous administration where it is desirable to form a depot of localized AAEs that is slowly released into the body by combining the AAEs at a concentration of 1 to 10% by weight with a carrier composed of sterile sesame oil with 2% w/w oleic acid USP. The solution is thoroughly mixed to ensure solubilization of the AAEs. The solution is then administered by subcutaneous injection as required.
6) Topical administration—AAEs may be formulated for topical administration by combining the AAEs at a concentration of 1 to 10% by weight with a carrier composed of 5% by weight Dow 245 fluid, 5% by weight Dow 5225C thickener, 5% by weight Dow 2051 formulation aid, 10% by weight AAEs with the balance water with or without preservatives, pH adjusting agents, perfumes or colorants as desired. The solution is thoroughly mixed to ensure solubilization of the AAEs. The solution is then administered by topical application as required.
7) Topical administration—AAEs may be formulated for topical administration by combining the AAEs at a concentration of 1 to 10% by weight with a carrier composed of 0.5% by weight Lubrizol Carbopol Ultrez 10, 0.5% by weight Carbopol 1382 thickener, 5% by weight AAEs with the balance water with or without preservatives, pH adjusting agents, perfumes or colorants as desired. The polymer is caused to gel by the addition of dilute sodium hydroxide solution raising the pH of the solution to between 5.5 and 7.5 as desired. The solution is thoroughly mixed to ensure solubilization of the AAEs. The solution is then administered by topical application as required.

To summarize:

The above-described experiments show how AAEs exert beneficial pharmacological effects. The experiments show that the AAEs work by modulating the non-covalent intermolecular interactions between various molecular species. This characteristic is manifested in different ways in each of the experiments.

Up until now, azelaic acid and its esters have been thought to have primarily antibacterial effects achieved by direct killing of bacteria. Azelaic acid has also been long known to have some degree of anti-inflammatory activity, but this effect was usually overshadowed by its strong irritancy due to its acidity. In clinical practice, the major dose limiting toxicity of azelaic acid was skin irritation.

What these experiments demonstrate is that the real mechanisms of action of the AAEs are much more complex and subtle than the prior art shows. The utility of the AAEs in medicine is extremely broad because all biological interactions operate at some level through non-covalent intermolecular interactions.

Cancers for instance have recently been found to send signals through the systemic circulation to immune cells residing in the bone marrow. These signals cause the bone marrow cells to leave the bones and travel to the corpus of the tumor. The immune cells then are instructed by the tumor to burrow into the mass of the tumor, where they are ‘enslaved’ by the tumor so that they can be used by the tumor to produce signaling molecules that prevent tumor cells from activating programmed cell death pathways, in essence immortalizing the tumor. Thus using AAEs to modulate, alter, cut off or decrease this signaling could prevent the tumor from recruiting bone marrow cells and thereby starve the tumor of factor essential for its growth and survival.

Another example of the benefits of treatment with AAEs can be shown in the case of toxin producing bacteria. Bacteria produce toxins to promote their survival. Mycobacterium ulcerans produces mycolactone. Mycolactone causes immune cells to become quiescent, disarming them by preventing them from making or responding to intercellular signaling molecules. The mycobacteria then invade, and multiply within the disarmed immune cells and then kill them when they have served their purpose. The bacteria then go on to another cycle of recruitment, infection and killing. The AAEs, by virtue of their ability to prevent these toxins from functioning, allow the immune system to avoid toxin-mediated damage, facilitating the processes of bacterial killing and clearance.

The AAEs are pharmacologically active.

Each of the individual AAEs has pharmacological activity that is unexpectedly different from the others esters, as is different from that of the parent acid.

Combinations of various esters can be selected to elicit desired biochemical responses in biological systems, with these combinations having complementary additive and or synergistic biological activity. The pharmacological activity of the AAE mixtures can thus be tailored to produce desired biological outcomes for the treatment of diseases and conditions wherein the diseases manifest as part of their pathophysiology abnormal changes in molecular signaling. The pathophysiology of diseases that can thus be moderated, mediated or otherwise altered by the application of selected azelaic acid esters having activities antagonistic to the pattern of signaling molecules characteristic of the disease.

The AAEs have antibacterial activity at relatively high concentrations, but they have important biological activity at concentrations well below the concentrations of esters that have antibacterial activity.

The biological activity of the AAEs is distinct from that of azelaic acid.

The AAEs modulate intra- and inter-cellular signaling.

The AAEs modulate pathogen sensing by modulating protein-protein interactions between innate and or exogenous molecular species.

The AAEs modulate the activity of membrane-associated proteins.

The AAEs modulate the activity of cytosolic proteins.

The AAEs modulate the activity of secreted, extracellular and intracellular proteins.

The AAEs exert their biological effects in part by modulating receptor mediated signaling.

The AAEs exert their biological effects in part by modulating the non-covalent interactions between biological molecules.

The AAEs exert their biological effects by modulating the interactions between exogenous and endogenous biological molecules.

The AAEs exert their biological effects by modulating the interactions between molecules that, as part of their mechanism of action, must form non-covalent multimeric molecular assemblies.

The AAEs exert their biological effects by modulating the physicochemical properties of lipid membranes.

The AAEs exert their biological effects by modulating the formation of intermolecular assemblies of membrane bound or associated biological molecules.

The AAEs exert their biological effects by modulating the physicochemical properties of the membrane micro-domains referred to as lipid rafts.

The AAEs exert their biological effects by modulating the formation of non-covalent assemblies of biological molecules associated with lipid rafts.

The AAEs exert their biological effects by modulating the non-covalent interactions of bacterial toxin protein subunits, particularly those toxins that attack or cross lipid membranes as part of their mechanisms of action.

It will be appreciated by those skilled in the art that changes could be made to the embodiments described above without departing from the broad inventive concept thereof. It is understood, therefore, that this invention is not limited to the particular embodiments disclosed, but it is intended to cover modifications that are within the spirit and scope of the invention.

The term “treat” or “treatment” means that the symptoms associated with one or more conditions mentioned above are alleviated or reduced in severity or frequency and the term “prevent” means that subsequent occurrences of such symptoms are avoided or that the frequency between such occurrences is prolonged.

The term “immune system” means the cells, tissues and various molecular species produced by those cells and tissues of the body that are primarily responsible for fighting infections, repairing damage due to trauma, forming and maintaining physical barriers that prevent the entry of pathogens, and repairing damage due to exposure to various toxic materials present in the environment.

The term “plasma membrane” and/or “cell membrane’ means the outermost physical barrier of a eukaryotic cell that separates the interior of the cell from the outside environment. The cell membrane is composed of lipids, phospholipids, proteins, polysaccharides, lipoproteins, membrane anchored glycoproteins, lipid anchored polysaccharides, glycolipoproteins and other molecular species.

The term “lipid raft” means a cell membrane microdomain, typically 10 to 200 nm in size, which is enriched in cholesterol and sphingolipids and plays host to a variety of cellular receptors and membrane associated proteins that perform essential cellular functions such as T cell antigen receptor signaling, insulin receptor signaling and others. There are at present thought to be two types of lipid rafts—planar rafts and caveolae.

The term “biological membrane” means a membrane forming a boundary between two regions of a biological system. Examples include cell membranes, bacterial cell walls, plant cell walls, nuclear membranes, vesicle membranes, the Golgi membranes, endoplasmic reticulum and the mitochondrial membranes.

The term “cytokine” broadly defined to encompass all soluble proteinaceous species having biological functions that are produced by cells into the intra- or extracellular milieus for the purpose of signal transduction. The term cytokine as used herein non-exclusively encompasses cytokines, chemokines, adipokines, growth factors, hormones, neuropeptides, and so on. The term is used in this way primarily to simplify this text.

The term “signaling molecule” means all molecular entities that are used in intercellular and intracellular biochemical signal transduction.

The term “biological molecule” means any molecule or ionic species that plays some role in or interacts with a biological system including cells, tissues or whole organisms.

The term “receptor” means any molecular entity present within an organism or any of its tissues or cells that interacts with any biological molecule, including signaling molecules, in such a way as to produce a subsequent alteration in the physiological state of the cells or tissues of a biological organism.

The term “intramolecular” means an interaction of any kind between two or more regions of a single covalently bonded molecule. An example of an intramolecular type interaction is that observed in transmembrane ion channels where regions of particular secondary structural motifs associate with each other non-covalently to produce a tertiary structural feature of the channel.

The term “intermolecular” means an interaction of any kind between two or more molecules. In the case of proteins these interactions give rise to quaternary structure of the interacting proteins.

The term “intracellular” means the region encompassed by the plasma membrane of a single cell and all molecular species present therein.

The term “intercellular” means all interactions occurring between two or more cells whether mediated by electrical impulses, through direct cell to cell contact interactions or through the agency of soluble molecules or ions.

The term “extracellular” refers to all regions outside of the plasma membrane of a cell.

1. A method of modulating markers in a tissue comprising administering to the tissue a composition comprising a mixture of azelaic acid esters (AAEs), wherein the markers comprise those associated with the activation of protein kinase C that are induced by exposure of the tissue to croton oil.

2. The method of claim 1, wherein the mixture comprises dimethyl azelate and diethyl azelate.

3. A method of modulating markers in a tissue comprising administering to the tissue a composition comprising a mixture of azelaic acid esters (AAEs), wherein the markers comprise inflammatory markers.

4. The method of claim 3, wherein the mixture comprises dimethyl azelate and diethyl azelate.

5. The method of claim 3, wherein the markers comprise interleukins.

6. The method of claim 5, wherein the interleukins comprise IL-4 and IL-10.

7. The method of claim 6, wherein IL-4 and IL-10 released from the tissue is increased.

8. The method of claim 6, wherein IL-4 and IL-10 in the tissue is depleted.

9. The method of claim 5, wherein the interleukins comprise IL-17 and IL-23.

10. The method of claim 9, wherein IL-17 and IL-23 released from the tissue is suppressed.

11. The method of claim 9, wherein IL-17 and IL-23 in the tissue is elevated.

12. The method of claim 1, wherein the method is performed in vitro.

13. The method of claim 1, wherein the method is performed in vivo.

14. A method of treating a subject having a disease associated with the activation of protein kinase C or inflammatory markers comprising administering to the subject a composition comprising a mixture of azelaic acid esters.

15. The method of claim 14, wherein the mixture comprises dimethyl azelate and diethyl azelate.

16. The method of claim 14, wherein the mixture is present in a range of from about 1 percent by weight to about 10 percent by weight of the composition.

17. The method of claim 14, wherein the composition is a topical formulation.

18. The method of claim 17, wherein the composition further comprises a carrier, a thickener, a pH buffering agent, and water.

19. The method of claim 17, wherein the composition further comprises one or more of a preservative, a perfume, or a colorant.
